(19) United States
(12) Patent Application Publication
(10) Pub. No.: US 2023/0175897 A1
(43) Pub. Date: Jun. 8, 2023

(54) APPARATUSES, KITS AND METHODS FOR TESTING WATER QUALITY

(71) Applicant: 9352-4585 QUÉBEC INC., Montreal (CA)

(72) Inventor: Claudio CARDINALE, Montreal (CA)

(73) Assignee: 9352-4585 QUÉBEC INC., Montreal (CA)

(21) Appl. No.: 17/839,421

(22) Filed: Jun. 13, 2022

Related U.S. Application Data
(63) Continuation of application No. 16/311,673, filed on Dec. 19, 2018, now abandoned.
(60) Provisional application No. 62/352,522, filed on Jun. 20, 2016.

(51) Int. Cl. G01K 13/02; G01N 1/20; E03C 1/04; G01N 33/18; E03B 7/07

(52) U.S. Cl. CPC G01K 13/02; G01N 1/20; E03C 1/0404; G01N 33/1853; E03B 7/07; E03B 7/072; G01K 13/026

(57) ABSTRACT

The present disclosure relates to apparatuses, kits and methods for testing quality. The water testing apparatus for analyzing at least one parameter and/or at least one substance from water flowing through a faucet and/or a water pipe comprises at least one sensor configured to directly contact said water flowing through said faucet and/or said water pipe, and an analyzer for analyzing said at least one parameter and/or at least one substance, said analyzer being in communication with said at least one sensor.

### CROSS-REFERENCE TO RELATED APPLICATION

The present application is a continuation application of U.S. Ser. No. 16/311,673, filed on Dec. 19, 2018, that is a 35 USC 371 national stage entry of PCT/CA2017/050750, filed on Jun. 20, 2017, and which claims priority to U.S. provisional application No. 62/352,522, filed on Jun. 20, 2016. These documents are hereby incorporated by reference in their entirety.

### FIELD OF THE DISCLOSURE

The present disclosure relates to apparatuses, kits and methods for testing water quality.

### BACKGROUND OF THE DISCLOSURE

Harmful contaminants such as heavy metals, pesticides and bacteria may infiltrate the local water supply system and find their way into homes and buildings, potentially creating an important public safety concern. Water testing kits are useful for assessing the quality of water and determining if the water is safe for use, especially when the water is intended for drinking, consumption or washing. Water testing kits can also provide other useful information on water such as water hardness. There remains however a need for economical water testing kits that are convenient and that can be readily installed on existing water plumbing and faucets. There is also a need for water testing kits that can provide information on the quality of the water in real-time.

It would thus be highly desirable to be provided with apparatuses, kits and methods that would at least partially address the disadvantages of the existing technologies.

### SUMMARY

According to an aspect of the present disclosure, there is provided a water testing apparatus for analyzing at least one parameter and/or at least one substance from water flowing through a faucet and/or a water pipe, the apparatus comprising:
at least one sensor configured to directly contact water flowing through the faucet and/or the water pipe; and
an analyzer for analyzing the at least one parameter and/or at least one substance, the analyzer being in communication with the at least one sensor.

According to another aspect of the present disclosure, there is provided a water testing kit comprising the water testing apparatus herein described and a faucet.

According to another aspect of the present disclosure, there is provided a water testing kit comprising the water testing apparatus herein described and a faucet.

According to a further aspect of the present disclosure, there is provided a method of monitoring and/or analyzing in real-time water quality flowing through a faucet and/or a water pipe, the method comprising:
installing to the faucet and/or the water pipe a water testing apparatus dimensioned to be at least partially in contact with the water, the apparatus configured for analyzing water quality;
carrying out an analysis of the water; and communicating results of the analysis.

According to another aspect of the present disclosure, there is provided a method of determining, in real-time, a presence or an absence of water contaminants flowing through a faucet and/or a water pipe, the method comprising:
installing to the faucet and/or the water pipe a water testing apparatus dimensioned to be at least partially in contact with the water, the apparatus configured for analyzing water quality;
carrying out an analysis of the water; and
communicating results of the analysis.

It has been found that the apparatuses, kits and methods of the present disclosure are effective for providing, in real time, awareness to the public of contaminants that may be contained in the local water supply systems, which may help in the prevention of certain illnesses due to ingestion or consumption of water containing certain types of contaminants.

### BRIEF DESCRIPTION OF THE DRAWINGS

In the following drawings, which represent by way of example only, various embodiments of the disclosure:

FIG. 1 illustrates a perspective view of a water testing apparatus comprising a coupling member according to one exemplary embodiment;

FIG. 2 illustrates a longitudinal partial cross-sectional view of the apparatus of FIG. 1;

FIG. 3 illustrates a transverse partial cross-sectional view the apparatus of FIG. 1;

FIG. 4 illustrates a front view of an aerator with digital LCD; according to an embodiment;

FIG. 5 illustrates a front view of an aerator with a code visual display; according to another exemplary embodiment;

FIG. 6 illustrates a front view of an aerator with an LCD screen showing three readings, according to another exemplary embodiment;

FIG. 7 illustrates an exploded front view of the aerator of FIG. 5;

FIG. 8 illustrates a perspective cross-sectional view of a two handle faucet with an analyzer;

FIG. 9 illustrates a transparent perspective view of a single handle faucet with an analyzer according to another exemplary embodiment;

FIG. 10 illustrates a perspective view of a faucet displaying two visual displays and installed on a sink; and

FIG. 11 illustrates a perspective view of the faucet displaying two visual displays. The faucet is connected to hot and cold water piping and the analyzer contained in the receptacle, both of which are concealed below the sink.

### DETAILED DESCRIPTION OF THE DISCLOSURE

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

In one aspect, there is provided a water testing apparatus for analyzing at least one parameter and/or at least one substance from water flowing through a faucet and/or a water pipe, the apparatus comprising:
at least one sensor configured to directly contact the water flowing through the faucet and/or the water pipe; and
an analyzer for analyzing the at least one parameter and/or at least one substance, the analyzer being in communication with the at least one sensor.

For example, the apparatus further comprises a receptacle for enclosing the analyzer therein.

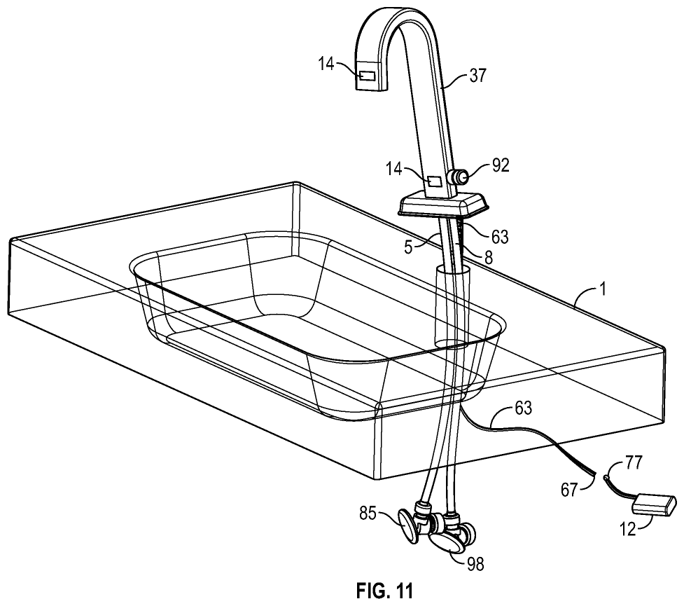

The analyzer, for example the analyzer comprised in a receptacle, may be located at various locations. For example, the analyzer may be positioned near the outlet of the faucet. For example, the analyzer may not be adjacent to the faucet, as shown in FIG. 11.

It will be understood that the receptacle, for example a chip box, can be in any shape or form and can be made of any suitable material. For example, as the water condensation may affect the integrity of the receptacle, the receptacle can be made of non-corrosive materials.

For example, the apparatus further comprises a coupling member dimensioned to receive the analyzer and dimensioned to be connected to the faucet and/or the water pipe.

For example, the coupling member comprises opposing ends suitable for sealing attachment to the faucet and/or water pipe.

For example, the coupling member can be attached to the faucet and/or water pipe by shark bite, compression, glue, soldering or any other type used in plumbing industry to join to elements together.

For example, when the apparatus comprises a coupling member, the analyzer may be located adjacent to a water pipe.

In some embodiments, the at least one sensor can be contacted with a hot water from a hot water pipe or with cold water from a cold water pipe or a mixture of hot and cold water, for example further downstream in the water piping and/or faucet where hot and cold water are mixed together.

For example, the apparatus further comprises a power source for powering the analyzer.

The person skilled in the art will understand that the analyzer can be powered by any suitable form of energy.

For example, the power source is a battery.

For example, the receptacle further comprises a battery compartment for enclosing the battery.

For example, the apparatus further comprises at least one visual display for displaying results of an analysis carried out by the analyzer.

It will be understood that the at least one visual display may be positioned at various places.

For example, the at least one visual display is disposed on the receptacle.

For example, the at least one visual display is disposed on the faucet, for example on the neck or body of the faucet.

For example, the at least one visual display is dimensioned to be connected to the faucet.

For example, the at least one visual display is dimensioned to be connected to the outlet of the faucet.

For example, the apparatus further comprises an annular member dimensioned to be connected to the outlet of the faucet.

It will be understood that the annular member, for example an aerator, can be connected to the outlet of the surface by various means, for example by screwing onto the faucet.

For example, the apparatus further comprises at least one visual display for displaying results of an analysis carried out by the analyzer, the at least one visual display being disposed on the annular member.

For example, the at least one sensor is dimensioned to be positioned adjacently to the outlet of the faucet and is configured to directly contact the water flowing through the faucet, the at least one sensor being at least substantially concealed by the annular member.

For example, the annular member is an aerator.

For example, the at least one sensor is dimensioned to be inserted into the coupling member via a bore.

It will be understood that more than one sensor may be included in the apparatus. For example, the apparatus comprises two sensors. For example, the apparatus comprises three, four or five sensors.

For example, the at least one sensor communicates with the analyzer via a wire.

For example, the apparatus further comprise an emitter connected to the analyzer, the emitter being suitable for communicating results of the analysis.

For example, the apparatus further comprises an emitter connected to the analyzer, the emitter being suitable for communicating results of the analysis to the at least one visual display.

For example, the apparatus further comprises an emitter connected to the analyzer, the emitter being suitable for communicating results of the analysis to a remote visual display.

For example, the remote visual display is chosen from a smart phone, a computer and a tablet.

It will be understood that the at least one visual display and the remote visual display can display different information according to the type of analysis carried out.

For example, the visual display can indicate parameters such as the water temperature and the water pH. The visual display can also indicate the hardness of water, as measured by quantifying for example the levels of calcium bicarbonate and magnesium bicarbonate.

For example, the visual display can indicate the presence, absence, concentration and level of substances herein described which are analyzed by the analyzer.

For example, the visual display can indicate the percentage of battery life.

For example, the visual display can provide a reading by color code.

For example, red (or any other type of visual symbol) can be associated to a water of poor quality or to non-potable or non-drinkable water. For example, green (or any other type of visual symbol) can be associated to excellent or good quality water and yellow (or any other type of visual symbol) can be associated with drinkable or potable water of low or medium quality.

For example, the visual display can comprised three readings providing water hardness, battery life and water temperature.

For example, the analyzing comprises detecting and/or quantifying the at least one parameter and/or at least one substance.

For example, the analyzing comprises detecting and/or quantifying at least one parameter chosen from temperature and pH.

For example, the analyzing comprises detecting and/or quantifying at least one substance chosen from minerals, metals and contaminants.

For example, the analyzing comprises detecting and/or quantifying at least one substance chosen from calcium, magnesium, calcium bicarbonate, magnesium bicarbonate, arsenic, barium, cadmium, chromium, lead, copper, mercury, selenium, nickel, thallium, antimony, and beryllium.

For example, the analyzing comprises detecting and/or quantifying at least one substance chosen from disinfectants, disinfection byproducts, inorganic chemicals, organic chemicals and radionuclides.

For example, the analyzing comprises detecting and/or quantifying at least one substance chosen from heavy metals.

For example, the analyzing comprises detecting and/or quantifying at least one substance chosen from microorganisms.

For example, the microorganisms are chosen from viruses, bacteria and protozoan parasites.

Another aspect herein described is a water testing kit, comprising the water testing apparatus herein disclosed and a faucet.

For example, the apparatus comprises a coupling member.

For example, the apparatus comprises an annular member.

For example, the apparatus comprises an aerator.

For example, the faucet further comprises a display visual disposed thereon for displaying results of an analysis carried out by the analyzer.

For example, the kit further comprises a cold water pipe and/or a hot water pipe.

For example, the kit further comprises a sink.

In a further aspect there is provided a method of monitoring and/or analyzing in real-time water quality flowing through a faucet and/or a water pipe, the method comprising:
installing to the faucet and/or the water pipe a water testing apparatus dimensioned to be at least partially in contact with the water, the apparatus configured for analyzing water quality;
carrying out an analysis of the water; and communicating results of the analysis.

Also provided herein is a method of determining, in real-time, a presence or an absence of water contaminants flowing through a faucet and/or a water pipe, the method comprising:
installing to the faucet and/or the water pipe a water testing apparatus dimensioned to be at least partially in contact with the water, the apparatus configured for analyzing water quality;
carrying out an analysis of the water; and communicating results of the analysis.

For example, the results are communicated to at least one visual display.

According to an aspect, there is provided herein a method of monitoring and/or analyzing in real-time water quality flowing through a faucet and/or a water pipe, the method comprising:
installing a water testing apparatus herein described;
carrying out an analysis of the water; and
communicating results of the analysis.

In a further aspect, there is provided a method of determining, in real-time, a presence or an absence of water contaminants flowing through a faucet and/or a water pipe, the method comprising:
installing a water testing apparatus herein described;
carrying out an analysis of the water; and
communicating results of the analysis.

The following examples are non-limitative and are used to better exemplify the materials and processes of the present disclosure.

### EXAMPLES

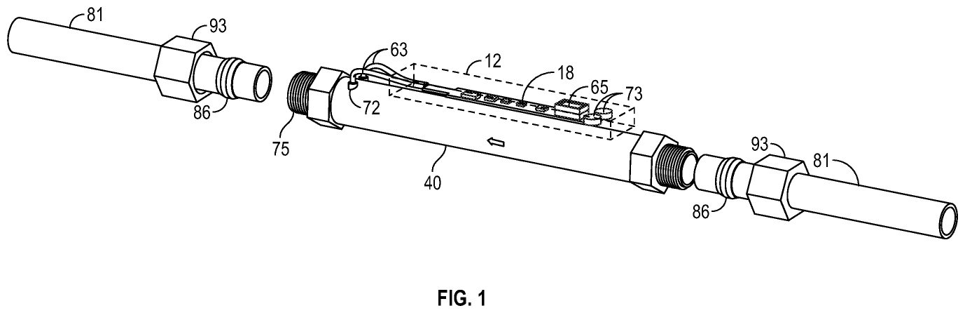

Referring now to FIG. 1, therein illustrated is a perspective view of a water testing apparatus for use with any cold water or hot piping 81. The piping 81 is inserted into the coupling member 40 then a nut 93 is tightened onto the connector 75 to form a tight seal between the water pipe 81 and the coupling member 40, allowing water to flow and make contact with the sensors 72. The sensors 72 transfer the signal through the wire 63 in the analyzer 18 and then give a reading on the LED screen 65 which here is powered by battery 73.

According to various exemplary embodiments, the primary opening 36 may be sized according to a size of the piping layout. The sensors 72 in which are inserted in the front of the coupling 40 will be in contact with the water flowing through 36 in which will touch the sensors 72 sending a signal through the wiring 63 which is connected to the analyzer 18 and then giving a reading on the LED screen 65.

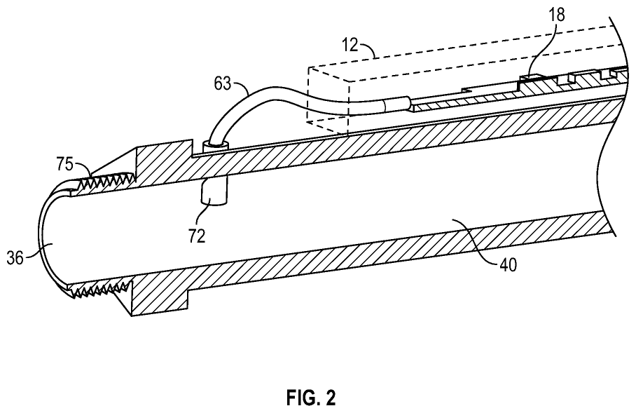

Referring now to FIG. 2, therein illustrated is a longitudinal partial cross-sectional view (view along the longitudinal axis) of the apparatus for use with any cold water or hot water piping. Once the water pipe is connected to the coupling member 40, a nut is threaded onto the connection 75 (male adapter) to tighten the seal which prevents the water flowing through from leaking near the opening 36. The primary opening 36 may be sized according to a size of the piping layout. The sensors 72 in which are inserted in the front of the coupling member 40 will be in contact with the water flowing through the opening 36 in which will touch the sensors 72, sending a signal through the wire 63 which is connected to the analyzer 18, and then giving a reading on the LED screen 65 (not shown in FIG. 2).

According to various exemplary embodiments, the sensors 72 can be made of any conductive material that can receive and transmit a reading.

According to various exemplary embodiments, as shown on FIGS. 1, 2 and 3, the connection 75 is a male adapter meant for compression. However, the connection can be made by any type of connecting means. Also the size of the coupling member can be any diameter.

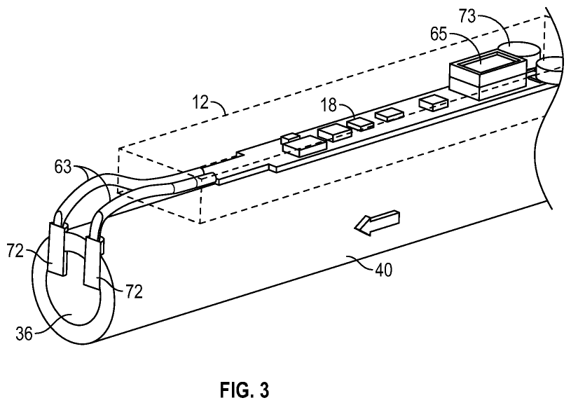

Referring now to FIG. 3, therein illustrated is transverse partial cross-sectional view (i.e. the cross-section view through the sensors 72) of the water testing apparatus to be connected to a water pipe (not shown). It is also shown that the sensors 72 are inserted into the coupling member 40 via bores. At least a portion of such an apparatus will thus be in fluid flow communication with the water pipe (not shown) and faucet (not shown). The apparatus includes a receptacle 12 portion that holds the analyzer 18, the LED screen 65 and a battery compartment 73.

According to various exemplary embodiments, as shown in FIGS. 1, 2 and 3 the receptacle (or encasement) 12 can be also made of any material respectively should be made of a none corrosive material for a reason of the condensation of the cold water can affect the receptacle 12.

According to various exemplary embodiments, the analyzer 18 that is held in the receptacle 12 can also be used to transmit a reading not only on a visual display screen 65 but also by Bluetooth or WIFI to a smart phone, tablet or computer.

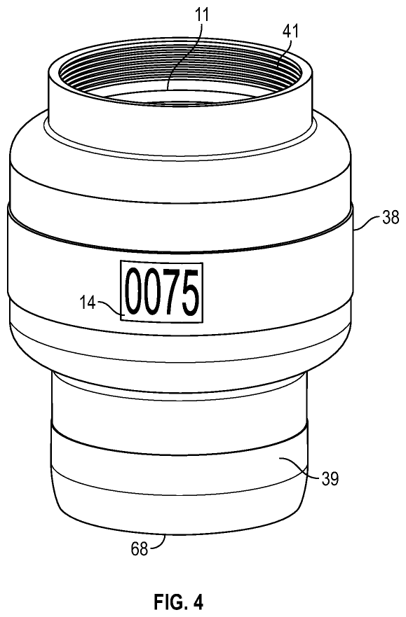

Referring now to FIG. 4, therein illustrated is a front view of an aerator 38 which is screwed onto a faucet by means of the female thread 41, in which the water flows from the portion of the aerator connected to the faucet 11 and through the screen 39 and exiting from 68. FIG. 4 also shows a digital reading 14 (shown as “0075”) being displayed.

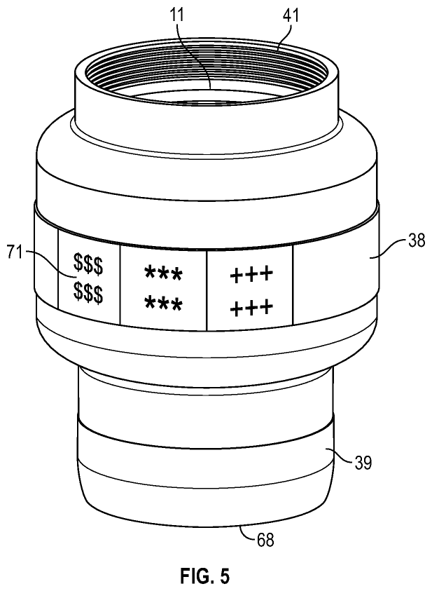

Referring now to FIG. 5, therein illustrated is a front view of an aerator 38 which is screwed onto a faucet by means of the female thread 41, in which when the water flows through the portion of the aerator connected to the faucet 11 and through the screen 39 and exiting from 68. FIG. 5 also shows a reading in code 71 (shown as “$$$”, “***” and “+++”) being displayed. Such code can of course be of various colors such as green, yellow and red to provide a user with an associated message regarding the quality of water. For example, “+++” or red (or any other type of visual symbol) can be associated to a water of poor quality or to non-potable or non-drinkable water. For example, “$$$” or green (or any other type of visual symbol) can be associated to excellent or good quality water and “***” or yellow (or any other type of visual symbol) can be associated with drinkable or potable water of low or medium quality.

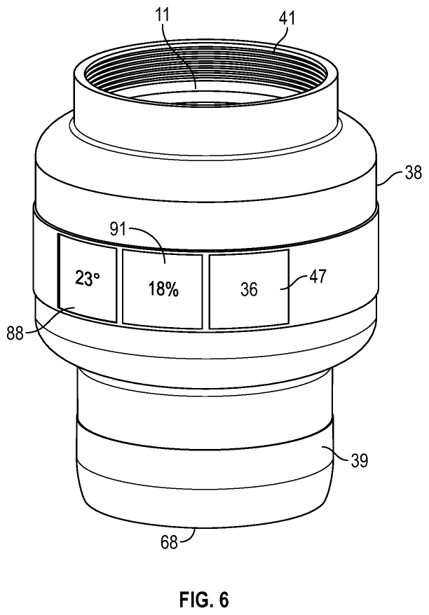

Referring now to FIG. 6, therein illustrated is a front view of an aerator 38 which is screwed onto a faucet by means of the female thread 41, in which when the water flows through the portion of the aerator connected to the faucet 11 and through the screen 39 and exiting from 68. FIG. 6 also shows a visual display reading in which three digital forms are displayed, namely hardness of the water 88, battery percentage 91 and water temperature 47.

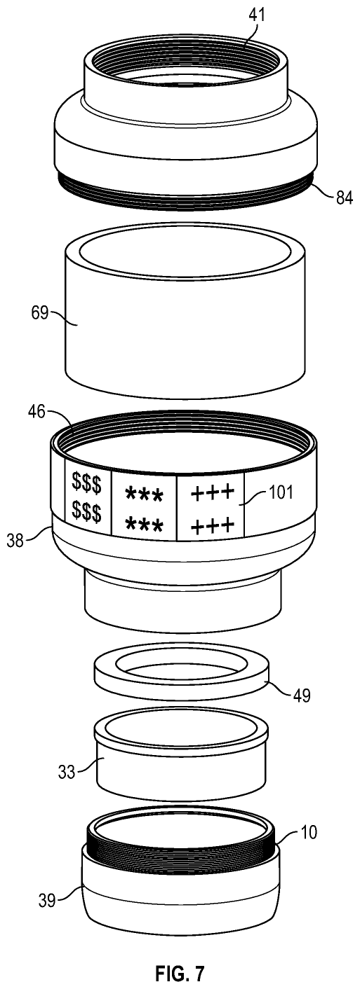

Referring now to FIG. 7, therein illustrated is an exploded view of the aerator 38. A female thread of an upper portion of the aerator 41 is screwed onto the faucet. The upper portion of the aerator also has a male tread 84 so that the body of the aerator 38 can be screwed on with a female thread 46. The filter 39 also has a male thread 10 that screws into the body of the aerator 38 and in between the body of the aerator 38 and filter 39 there is a rubber gasket 49 to secure against leaks and a screen-flow director 33. Further, between the female thread of the body of the aerator 46 and the male thread of the upper portion of the aerator 84 there is provided a ceramic module 69 that can be used in any material to send out readings to the aerator 38.

According to various exemplary embodiments, referring to FIGS. 4, 5, 6 and 7, all the aerators 38 work in a similar way in that the aerator 38 can be screwed onto any faucet of a household or building, thus giving a reading directly on the aerator 38 or by Bluetooth or WIFI to a smart phone, tablet or computer. Materials may also vary depending on the construction of the aerator 38.

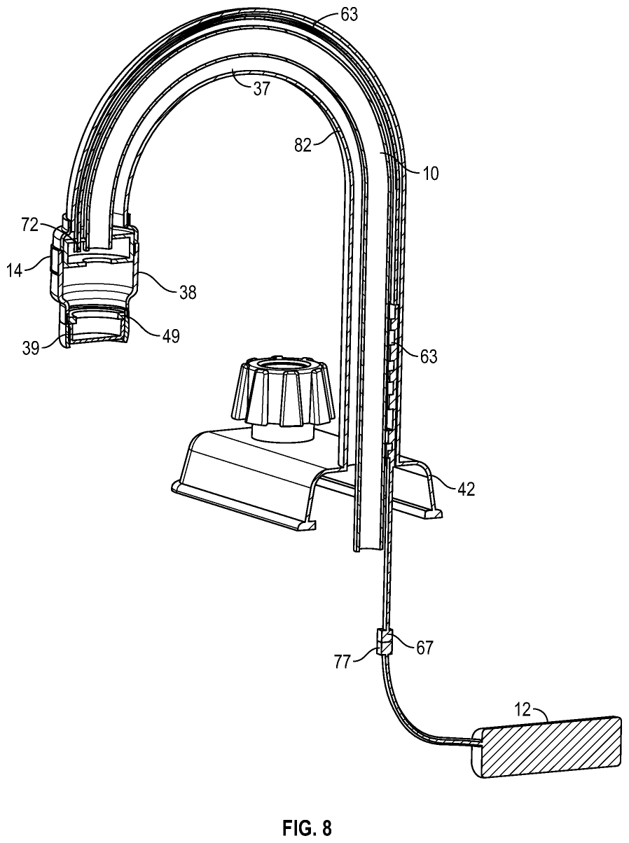

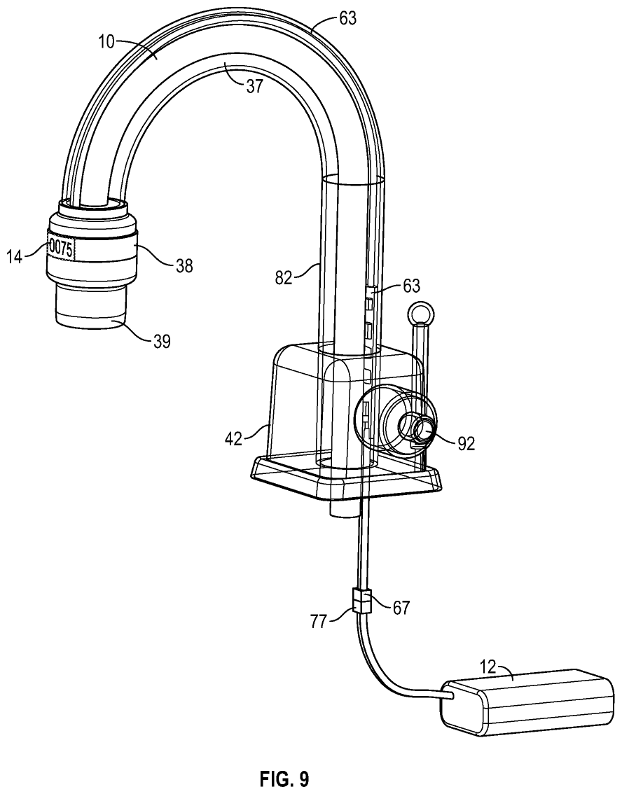

Referring now to FIGS. 8 and 9, therein illustrated are two perspective views. FIG. 8 is a perspective cross-sectional view of a two handle faucet and FIG. 9 is a transparent perspective view of a single handle faucet. In both FIG. 8 and FIG. 9, the water pipe 10 runs in the center of the body of the faucet 82 (also referred to as the empty space of the faucet 37). Once the faucet 82 is installed and the connectors 67 and 77 are plugged together to the receptacle 12 containing the analyzer, the wire 63 will also run its way through the empty space of the faucet 37 until its desired spot of the sensors 72 that will give a reading to the display screen 14 once the water flows through the aerator 38 and out of the filer 39, further having a secure gasket in place 49 to prevent leaking. FIG. 9 shows a similar installation except that the faucet is a single handle faucet 92.

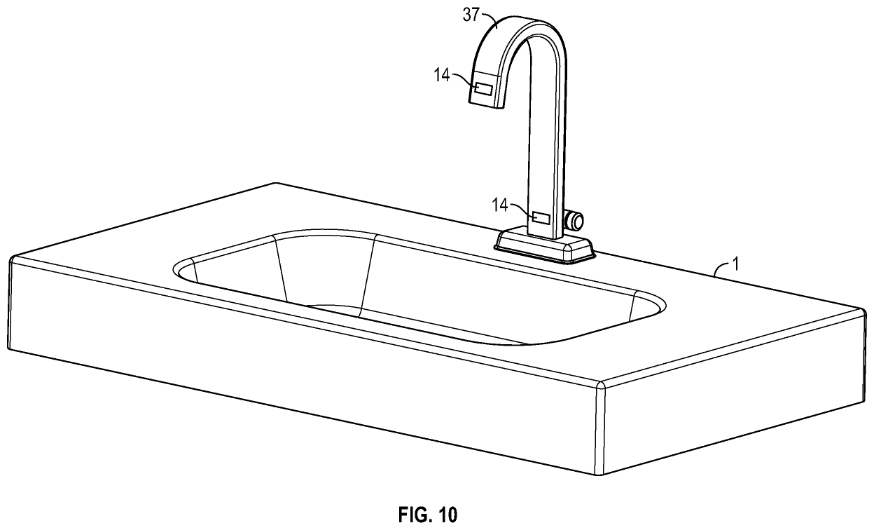

Referring now to FIG. 10, therein illustrated is a perspective view of the faucet 37 installed on a sink 1 showing that the digital screen 14 can be installed anywhere that is desired.

Referring now to FIG. 11, therein illustrated is a perspective view of the faucet 37 being installed on a sink 1 by passing the hot water pipe 5, the cold water pipe 8 and the wire 63 in the hole of the sink 1. Once the three parts are passed in through the hole of the sink 1 the hot water pipe 5 will connect to hot water valve 85 and the cold water pipe 8 will connect to the cold water valve 98. At the end of the wire 63 there can be a male or female adapter 67 that will fit in with the male or female adapter of 77 leading to the analyzer 18 enclosed in the receptacle 12 to be able to send a reading to the LED screens 14 upon opening of the faucet by the handle 92 in which in this figure is a single handle.

According to various exemplary embodiments, as shown on FIGS. 8, 9, 10 and 11, it will be understood that this technology can be used on any faucet construction or fabrication and may differ from faucet to faucet. Further, faucets may also provide a reading by Bluetooth or WIFI that can be emitted onto a smart phone, tablet or computer.

The scope of the claims should not be limited by specific embodiments and examples provided in the disclosure, but should be given the broadest interpretation consistent with the disclosure as a whole.

1-43. (canceled)

44. A method of monitoring and/or analyzing in real-time water quality flowing through a faucet and/or a water pipe, said method comprising:
installing to said faucet and/or said water pipe a water testing apparatus dimensioned to be at least partially in contact with said water, said apparatus configured for analyzing water quality;
carrying out an analysis of said water; and
communicating results of said analysis.

45. A method of determining, in real-time, a presence or an absence of water contaminants flowing through a faucet and/or a water pipe, said method comprising:
installing to said faucet and/or said water pipe a water testing apparatus dimensioned to be at least partially in contact with said water, said apparatus configured for analyzing water quality;
carrying out an analysis of said water; and
communicating results of said analysis.

46. The method of claim 44, wherein said results are communicated to at least one visual display.
